(19) United States
(12) Patent Application Publication
(10) Pub. No.: US 2019/0360019 A1
(43) Pub. Date: Nov. 28, 2019

(54) CLEAVAGE EVENT TRANSDUCTION METHODS AND PRODUCTS

(71) Applicant: Universal Biosensors Pty Ltd, Rowville, Victoria (AU)

(72) Inventors: Alastair M. HODGES, Blackburn South (AU); Ronald CHATELIER, Bayswater (AU)

(21) Appl. No.: 16/461,705

(22) PCT Filed: Nov. 16, 2017
(86) PCT No.: PCT/AU2017/051264

§ 371 Date: May 16, 2019

Related U.S. Application Data
(60) Provisional application No. 62/423,087, filed on Nov. 16, 2016.

(51) Int. Cl. C12Q 1/37

(52) U.S. Cl. CPC C12Q 1/37; C12Y 304/21004

(57) ABSTRACT

Disclosed herein are methods and products for the incorporation of an enzyme mediated reaction step and/or cascading amplification methods that are activated via a molecule cleavage event and methods and products for cleavage detection in assays where a binding event can be used to selectively detect the analyte of interest.

### CROSS REFERENCE TO RELATED APPLICATIONS

This application claims the benefit of U.S. Provisional Application No. 62/423,087, filed Nov. 16, 2016, entitled CLEAVAGE EVENT TRANSDUCTION METHOD. The entire contents of the foregoing are hereby incorporated by reference herein.

### BACKGROUND TO THE INVENTION

In some sensing applications it is desirable to be able to detect when cleavage of a molecule occurs. In prior art methods and devices this can be difficult as often there is either only a small or no detectable change in the properties of the sample being assayed. Sensing a cleavage event can be useful to directly detect an analyte that itself cleaves a molecule, or indirectly as part of a chemistry reaction chain between the analyte of interest and the readout signal. An example in the prior art of a direct detection is the detection of thrombin that is generated as part of blood clotting reactions. In prior art methods and devices, thrombin is detected, for example, by cleaving a substrate that releases an electroactive or coloured species. An example of an indirect detection in the prior art is where DNA or RNA that it is to be detected hybridizes to other strands of DNA to form a complex, termed an MNAzyme, that can cleave a substrate DNA strand containing an RNA bond. The cleavage of the substrate DNA can be detected, for example, by the separation of a fluorescent and quencher group causing a fluorescence signal to be generated.

These prior art methods can suffer from lack of sensitivity when the signal generated at the desired concentration of the analyte species is too small to easily detect. It is thus desirable to find ways to amplify this signal. A traditionally used method for chemically amplifying a signal is to have an enzyme-mediated reaction as part of the signal generation chemistry. The enzyme can cycle many times and thus produce multiple molecules in response to the presence of a single molecule or event occurring. Reaction schemes that produce a cascading increase in the signal generated are particularly advantageous in amplifying the signal from a low concentration of analyte.

The instant invention seeks to overcome this deficiency in the prior art by providing molecular entities and methods for the effective incorporation of an enzyme-mediated reaction step and/or cascading amplification methods that are activated via a molecule cleavage event. Also disclosed is a novel method of utilizing the invention or other cleavage detection methods in assays where a binding event is used to selectively detect the analyte of interest.

### SUMMARY OF INVENTION

Disclosed herein are molecular entities and methods for the incorporation of an enzyme-mediated reaction step and/or cascading amplification methods that are activated via a molecule cleavage event and methods and products for cleavage detection in assays where a binding event can be used to selectively detect the analyte of interest.

Some embodiments of the invention relate to a molecular entity that can include an enzyme and an enzyme inhibitor complex. The enzyme inhibitor complex can include an inhibitor portion joined to a linker portion. The linker portion can be capable of being tethered to the enzyme and can be capable of being cleaved by a cleaving agent. When the linker portion is tethered to the enzyme, activity of the enzyme can be inhibited. When the linker portion is cleaved, activity of the enzyme can be increased. Activity of the enzyme can be reversible.

Some embodiments of the invention relate to a molecular entity for detecting presence of a cleaving agent. The molecular entity can include an enzyme joined to a reversible enzyme inhibitor by a linker that cleaves in the presence of the cleaving agent.

In some embodiments, the linker portion can include a cleavage site, a distal side and a medial side. The medial side and distal side are at opposite sides of the cleavage site. The inhibitor portion can be linked to the linker portion at a position on the medial side and the linker portion can be capable of being linked to the enzyme by a reactive species at a position on the distal side.

In some embodiments, the linker portion can include at least two cleavage sites, a middle region and at least two sides distal to the middle region. A cleavage site can be positioned between each distal side and the middle region. The inhibitor portion can be linked to the linker portion in the middle region and the linker portion can be capable of being linked to the enzyme by a reactive species at a position on the distal sides. In some embodiments, the reactive species can be capable of forming a covalent bond with the enzyme. The reactive species can be capable of forming a covalent bond with the enzyme either directly or via a crosslinking agent. In some embodiments, the distal end of the linker portion can link to the enzyme via the application of UV light, a crosslinking agent, an activating agent, catalyst, heat, or the like.

In some embodiments, the inhibitor portion can associate with the enzyme at a concentration of enzyme inhibitor complex mixed with a concentration of enzyme. The concentrations can be higher than the concentration of inhibited enzyme complex to be used in an assay reagent.

In some embodiments, the enzyme can be a redox enzyme, a cleavage enzyme, or the like.

In some embodiments, the enzyme inhibitor complex can include a linker amplifying portion. The linker portion can be capable of being cleaved by a cleaving agent. Cleavage of the linker portion can result in (i) activation of the enzyme and (ii) formation and activation of the linker amplifying portion that can be capable of cleaving a second linker portion or when joining with other components can be capable of cleaving a second linker portion. In some embodiments, the linker amplifying portion can form a DNAzyme or one or more portions of an MNAzyme when activated. In some embodiments, the cleaving agent can be a target analyte and wherein the activity of the enzyme generates a readout signal.

Some embodiments of the invention relate to a method for detecting a cleavage event in a molecule including: (a) providing a molecular entity that can include an enzyme and an enzyme inhibitor complex with a linker portion in a state wherein a linker portion is tethered to the enzyme and activity of the enzyme is inhibited; (b) subjecting a test sample to (a); and (c) detecting a device readable signal. Detection of a device readable signal can be indicative of a cleavage event. In some embodiments, the cleavage event can be indicative of the presence of an analyte in the test sample. In some embodiments, the entity of (a) can be contained in a solution, on a test strip, or the like. In some embodiments, the detecting step can include detecting changes in electrical voltage, electrical current, optical absorbance, colour, fluorescence, chemiluminescence, or the like.

Some embodiments of the invention relate to a kit for detecting a cleavage event that can include: (a) one or more reaction chambers; and (b) an assay reagent comprising an enzyme complex in a state wherein a linker portion is tethered to the enzyme and activity of the enzyme is inhibited. The assay reagent can be in solution form, dry form, or the like.

Some embodiments of the invention relate to a molecular entity complex that can include: (a) a carrier, and (b) an amplifying complex. The amplifying complex can include a linker portion tethered to the carrier in one or more positions. When the linker portion is linked to the carrier, activity of the amplifying complex can be inhibited. When the linker portion is cleaved, activity of the amplifying complex can be increased. In some embodiments, the carrier can be macromolecule, beads, rods, or the like. In some embodiments, the linker portion can be linked to the carrier by a covalent bond.

Some embodiments of the invention relate to a kit for detecting a cleavage event using a molecular entity that can include: (a) one or more reaction chambers; and (b) an assay reagent including an inhibited enzyme complex.

Some embodiments of the invention relate to a method for detecting a cleavage event including: (a) subjecting a test sample to a solution comprising a molecular entity comprising an enzyme and an enzyme inhibitor complex; (b) adding a molecular complex including a carrier and an amplifying complex when activity of the amplifying complex is inhibited; and (c) detecting a device readable signal. Detection of a device readable signal can be indicative of a cleavage event.

Some embodiments of the invention relate to a method for detecting a cleavage event that can include: (a) subjecting a test sample to a solution including substrate for a DNAzyme or MNAzyme that can include a fluorescent group and a quencher group; (b) adding a molecular complex including a carrier and an amplifying complex when activity of the amplifying complex is inhibited; and (c) detecting a fluorescence. Detection of fluorescence can be indicative of a cleavage event.

Some embodiments of the invention relate to a molecular entity that can include at least one binding moieties linked to one or more DNA strands. At least one of the DNA strands can include a DNAzyme or a component of an MNAzyme. In some embodiments, the one or more DNA strands can be dissociated from the molecular entity in the presence of complementary DNA or a temperature elevation in a detection chamber. In some embodiments, a molecule binding to the binding moiety can result in the molecular entity being present in a detection chamber.

In some embodiments, one or more branch DNA strands can be linked to a binding moiety via a DNA backbone. In some embodiments, the binding moiety can be an antibody. In some embodiments, one or more branch DNA strands can be linked to a DNA backbone. In some embodiments, DNAzymes or a component of MNAzymes can be linked to the branch DNA strands.

Some embodiments of the invention relate to a method for detecting a binding event that can include (a) subjecting a test sample to a solution comprising a molecular entity including at least one binding moieties linked to one or more DNA strands; (b) separating the molecular entities bound via the binding moiety from the molecular entities not bound via the binding moiety; (c) adding the bound or the unbound molecular entities to a substrate capable of a producing a device readable signal; and (d) detecting a device readable signal. Detection of a device readable signal can be indicative of a binding event comprising the binding moiety.

Some embodiments of the invention relate to a kit for detection of a cleavage event that can include (a) a first reaction chamber and a second reaction chamber; (b) an assay reagent comprising a molecular entity including at least one binding moieties linked to one or more DNA strands; and (c) an assay reagent comprising at least one of an inhibited enzyme complex, an amplifying complex, a DNAzyme or MNAzyme substrate comprising a fluorescent group and a quencher group. The first reaction chamber can be for forming a mixture of the test sample and (b). The second reaction chamber can be for detection of a device readable signal in the mixture.

In a first embodiment, a species that is capable of reversibly inhibiting the activity of an enzyme (inhibitor) is linked to one or more molecules or other species that can be cleaved in the presence of the analyte of interest (linker). A construct, comprising a linker portion and an inhibitor portion, is herein termed the inhibitor complex. The inhibitor complex can be mixed with an enzyme at relatively high concentration such that the inhibitor complex self-assembles to the enzyme and inhibits its activity. In some embodiments, a region of the linker portion distal to the inhibitor portion can comprise a group that can react with the enzyme to form a covalent or other suitably strong bond to the enzyme. During or after the inhibitor complex has self-assembled to the enzyme, the distal reactive group can be optionally activated causing it to form a bond to the enzyme. In this fashion, the inhibitor complex is tethered to the enzyme at at least one point via the distal reactive group and at at least a second point via the assembly of the inhibitor portion of the inhibitor complex to the enzyme or via a second linker portion. This forms an inhibited enzyme construct (see FIG. 1).

In some embodiments, a solution containing the inhibited enzyme construct can be purified to remove extraneous species as desired. To use the inhibited enzyme construct in a sensing application, a solution containing the inhibited enzyme construct can be introduced into a sensing system and either used as a liquid or the solution dried to leave a dry inhibited enzyme construct. When in use in an assay, the concentration of the inhibited enzyme construct can be lower than the concentration used when it was formed, for example, 10 to 100000, for example, 10, 15, 20, 25, 30, 35, 40, 50, 75, 100, 200, 300, 400, 500, 1000, 2000, 3000, 4000, 5000 up to 10000 times lower. In some embodiments, due to the tethering of the inhibitor complex to the enzyme via the linker, the local concentration of the inhibitor remains high, thus leading to the inhibitor continuing to inhibit the enzyme activity. In some embodiments, if the linker is cleaved due to the presence of an analyte, the inhibitor is free to dissociate from the enzyme due to the relatively low concentration of the inhibitor in the bulk solution. When the linker is cleaved, the inhibition of the enzyme is reversed and the enzyme becomes active, whereupon it can generate multiple species from its substrate to ultimately generate a device readable signal. In this fashion, the cleavage event selectively results in the activation of an enzyme that can be used as part of generating a device readable signal.

In a second embodiment, the linker portion of the enzyme inhibitor complex can comprise a linker amplifying portion, wherein the amplifying portion is activated upon cleavage of the linker. In some embodiments, the linker amplifying portion is not in the active configuration when joined to the enzyme, but when the linker is cleaved, it is liberated from the enzyme and becomes active. The activity of the amplifying portion includes the ability to cleave intact linkers either by itself or when in combination with other species. In this way, the amplifying portion is able to generate an exponential increase in the rate of cleavage events occurring and concentration of cleavage agents over time, after initiation by an initial cleavage event. The cleavage agents formed can serve to activate the enzyme in the inhibited enzyme construct according to embodiments described herein to generate a device readable signal (see FIG. 2).

In a third embodiment, the inhibitor complex according to the second embodiment of the invention is not required to be bound to or inhibit an enzyme. In this embodiment, the inhibitor complex can be bound to a carrier that serves to prevent the amplifying portion of the linker from being active in the bound state. The complex associating with the carrier is termed the amplifying complex (see FIG. 3). According to this embodiment the exponential growth in the concentration of cleavage agents proceeds as it does in the second embodiment, but in this embodiment, the cleavage agents generate a readable signal by cleaving a separate signal generation species. The separate signal generation species can be the inhibited enzyme construct according to the first embodiment of this invention. Alternatively, it can be another species, such as a substrate for a DNAzyme or MNAzyme that comprises a fluorescent group and a quencher group such as is known in the prior art, where the fluorescent group begins to fluoresce when the substrate is cleaved to separate it from the quencher group. This embodiment can be useful, for example, if it is difficult to satisfy all the required characteristics of the second embodiment simultaneously. This embodiment allows the required characteristics for the amplifying complex to be optimised separately from the characteristics required to generate a readable signal.

In a fourth embodiment, a method whereby detection of a cleavage event can be linked to a binding event is provided. In this embodiment, one or more DNA strands can be attached to a binding moiety such as an antibody. At least one and preferably multiple copies of a facilitator DNA are preferably hybridized to DNA attached to the binding moiety. This is here termed a label construct. In the presence of the analyte of interest, label construct is bound to a binding partner, leading to a change in the ratio of free to bound label construct. Either the remaining free or the bound label construct can be exposed to conditions where the hybridized DNA in the label construct can act as a cleaving construct, or be used to complete the assembly of a cleaving construct (see FIG. 4). The cleaving construct can then serve to activate the enzyme in the inhibited enzyme construct according to the first embodiment, or the carrier associated amplifying construct according to the second embodiment, or use other methods such as those described in the prior art to generate a device readable signal.

Also disclosed are devices suitable for use with the molecular entities disclosed herein and/or for practicing the disclosed methods.

### BRIEF DESCRIPTION OF THE DRAWINGS

FIG. 1 shows a schematic of an exemplary first embodiment of the invention, showing the assembly of the inhibited enzyme complex and the de-inhibition of the enzyme in an assay.

FIG. 2 shows a schematic of an exemplary second embodiment of the invention using Partzymes, showing the assembly of the inhibited enzyme complex with amplifying portions included in the linker, the de-inhibition of the enzyme in an assay and formation of an amplification MNAzyme.

FIG. 3 shows a schematic an exemplary third embodiment of the invention using Partzymes as part of the linker joined to a carrier, showing the formation of an amplification MNAzyme.

FIG. 4 shows an exemplary fourth embodiment of the invention, showing multiple facilitator DNA copies hybridized to branch and backbone DNA, with the backbone DNA joined to an antibody.

### DETAILS OF INVENTION

There are multiple methods known in the prior art for detecting a signal generated as a result of the presence and/or concentration of an analyte of interest. All these methods have advantages and disadvantages. Of particular interest for some embodiments disclosed herein are methods that incorporate an enzyme in the signal generation pathway to amplify the signal that can be generated from the analyte over the range of concentration required to have a useful assay for that analyte. These enzymes can be ones that directly generate a species that can be detected by a detection device and converted to an output, or can be ones that generate a species that feeds into further reactions that ultimately result in the generation of a species that can be detected by a detection device. The detection signal generated from the detection species can be in many forms, for example, it can be an electrical voltage or electrical current, a colour or turbidity that changes the characteristics of a transmitted or reflected light signal, an emitted light signal via for example fluorescence, or chemiluminescence. For simplicity, the instant disclosure will focus on a subset of the possible enzyme types that are capable of generating a species that can be detected, but it will be well understood by those of skill in the art that embodiments of the invention disclosed herein applicable to any application where an enzyme is present in the signal generation pathway.

In some embodiments of the invention disclosed herein, enzymes that generate, either directly or indirectly, an electroactive species are used. An enzyme reacted substrate can be detected directly at an electrode or mediators can be used to convert the products of the enzyme reaction to electroactive species. Two such classes of enzymes known in the prior art are redox enzymes or cleavage enzymes. The former oxidise or reduce their substrate and either form an electroactive species directly from the substrate or via a mediator. The latter cleave a substrate to generate an electroactive species. As well as electroactive species, these enzymes can also typically produce coloured species in a similar manner, where the absorption of particular wavelengths of light can be used to detect the species generated by the enzyme action.

Non-limiting examples of suitable redox enzymes are glucose oxidase, FAD dependent glucose dehydrogenase, PQQ dependent glucose dehydrogenase, lactose oxidase, and other enzymes that are capable of generating an electroactive species either directly or via a mediator. Non-limiting examples of non-redox enzymes that can also be suitable are thrombin, trypsin, and other enzymes that can cleave a substrate. These can be used to generate an electroactive, coloured, fluorescent or other detectable species by cleaving for example a peptide bond to liberate the detectable species.

A suitable inhibitor for embodiments of the invention disclosed herein includes one that can associate with the enzyme in some way and upon association prevent or significantly decrease the activity of the enzyme.

The inhibitor can also include at least one linker, or be able to be joined to at least one linker. When joined to the inhibitor, the linker is capable of being cleaved by the cleavage mechanism related to the presence of the analyte and contains a portion that is capable of being joined to the enzyme. The inhibitor complex can include one, two or more linkers to form one or more cleavable links to the enzyme. A possible advantage of having more than one linker is to more firmly tether the inhibitor portion and thus increase its inhibiting ability. Whether one, two or more linkers are optimal depends upon the strength of binding of the inhibitor to the enzyme and its inhibition strength, as well as the rate at which the linkers can be cleaved to de-inhibit the enzyme. In general, more linkers can be favourable for less strongly binding inhibitors.

The exact nature of the linker depends upon the cleaving species. The cleaving species can be a protein based enzyme, a DNAzyme or MNAzyme as known in the prior art, or other species capable of cleaving a substrate, wherein the linker includes a substrate for the chosen cleaving species. For a protein based enzyme such as a protease, the linker can include a peptide of the appropriate sequence of amino acids. For a DNAzyme or MNAzyme the linker can include a sequence of DNA containing at least one RNA bond that can be cleaved by the DNAzyme or MNAzyme.

The inhibitor complex can be joined to the enzyme via a tethering group that is preferably distal to the enzyme inhibitor portion of the inhibitor complex such that the linker cleavage site lies between the enzyme inhibitor portion and the tethering group. The function of this group is to tether the inhibitor complex to the enzyme. If there are multiple linkers comprising the complex, then a tethering group can preferably be present on each linker. The tethering group can be capable of forming a covalent bond to the enzyme or a non-covalent bond. Preferably, but not necessarily, the joining of the tethering group to the enzyme can be triggered by an external stimulus. Non-limiting examples of suitable tethering groups that can form a covalent attachment in response to an external stimulus include UV activated crosslinkers. An example of such a group is the ATFB (4-Azido-2,3,5,6-Tetrafluorobenzoic acid, from Life Technologies). Under UV light the aryl azide group undergoes C—H bond insertion on the enzyme to form a C—N bond. The succinimidyl ester of ATFB can be used to join this group to a primary amine group on the linker if desired, to join the tethering group to the rest of the inhibitor complex.

Apart from activation by UV light, other possible external triggering mechanisms include, without limitation, the addition of a crosslinking reagent, activating reagent, catalyst, or the application of heat. An example of a crosslinking reagent that can be added is 1-Ethyl-3-[3-dimethlyaminopropyl]carbodiimide hydrochloride (EDC). This reagent can couple a carboxyl group to a primary amine. EDC can be added to the mixture of inhibitor complex and enzyme prior to, during, or after the inhibitor complex has assembled to the enzyme. In some embodiments, EDC, or other suitable crosslinking reagent, can be added after the inhibitor complex has been allowed to self-assemble to the enzyme. Once added, the EDC can, for example, couple a primary amine on the distal end of the inhibitor complex to a carboxyl group on the enzyme. Alternatively, a carboxyl group on the distal end of the inhibitor complex can be coupled to a primary amine on the enzyme.

An example of a tethering group that is not triggered by an external stimulus and does not form a covalent bond is an aptamer that is designed to bind to the enzyme with high affinity.

There are known inhibitors in the prior art for enzymes that can be used in embodiments disclosed herein. For example, Mannocci et. al. in Bioconjugate Chemistry, 2010 volume 21, pp. 1836 to 1841, which is hereby incorporated by reference in its entirety, disclose inhibitors for trypsin that can form a basis for inhibitor complexes useful in embodiments herein. This paper describes various molecules designed to inhibit trypsin that have been joined to DNA as a mechanism for being able to conveniently identify the molecules that show affinity for trypsin. These are unsuitable in the form disclosed, but can be made suitable by at least replacing the DNA sequences disclosed with a sequence that is suitable as a linker in the current invention. Additional modifications can also be made to the inhibitor molecule and/or the DNA to be able to modify the DNA with a distal tethering group and to be able to attach the DNA correctly to the inhibition portion, either with prior or subsequent attachment of the tethering group. For example, the distal end of the DNA to the inhibitor portion can be modified with an amine group and proximal end of the DNA with a group suitable for “click” chemistry, as is known in the prior art. For example the proximal end of the DNA can be modified with an azide, alkyne, TCO (trans-cyclooctene), tetrazine or other suitable groups. The carboxylic acid group on the inhibitor molecules such as disclosed in Mannocci et. al., which is hereby incorporated by reference in its entirety, can be modified with the click chemistry conjugate pair of the modifier on the DNA to form a bond between the inhibitor molecule and the DNA. Alternatively, the proximal end of the DNA could be modified with a thiol group and with a primary amine group on the distal end and the inhibitor portion comprise a maleimide group that can react with the thiol group to join the inhibitor portion to the DNA. The primary amine on the distal end of the DNA can react with a succinimidyl ester to join the tethering group to the DNA.

To form the inhibited enzyme complex, the enzyme is mixed with the inhibitor complex, whereupon as least the inhibitor portion of the inhibitor complex will self-assemble to the enzyme. The concentration of the enzyme and inhibitor complex in this mixture is high enough to ensure that a high proportion of the enzyme will be associated with an inhibitor complex. Once the association is at the desired point an optional external stimulus can be applied to cause the tethering groups to join to the enzyme, thus anchoring the inhibitor complex in place. This sequence can be repeated one or more times if it is desired to inhibit enzymes that were not inhibited in previous rounds of this reaction. If an external stimulus is not used to tether the inhibitor complex to the enzyme, the tethering groups will be designed to associate with the enzyme when the inhibitor portion binds to the enzyme.

In some embodiments, after formation of the inhibited enzyme complex, it is preferable that it be purified of extraneous species, such as unbound inhibitor complex, uninhibited enzyme or other reactants added as part of the formation process. For example, unbound inhibitor complex can be separated from the inhibited enzyme construct on the basis of the size of the species. Enzyme not bound to an inhibitor complex can be separated from the inhibited enzyme construct by selectively binding the inhibitor complex portion of the inhibited enzyme construct to a binding medium to separate it from the enzyme without inhibitor complex. This can include conventional separation means such as filtration, chromatography, centrifugation or magnetic separation, and the like. After any desired purification has been performed, the inhibited enzyme complex is ready to be used in assays.

For use in an assay the inhibited enzyme complex is diluted by preferably between 5 and 100000 times, or more preferably between 10 and 10000 times, or between 100 and 5000 times, or between 500 and 1000 times. Due to the presence of the at least one tether comprising the tethering group and the linker, the inhibitor will not dissociate from the enzyme to a significant extent following this dilution process. In this context, significant dissociation means a degree of dissociation that is large enough for the enzyme to produce an unacceptably large assay signal when the tethers are intact. When the linkers are cleaved in the assay, the cleaved inhibitor complex will dissociate to a substantial extent from the enzyme due to the dilution of the inhibited enzyme complex, de-inhibiting the enzyme in the process. In this context, substantial dissociation means that the cleaved inhibitor complex is dissociated sufficiently that the enzyme can produce an acceptably large amount of signal generating species in the presence of the analyte of interest, over the range of analyte concentration of interest.

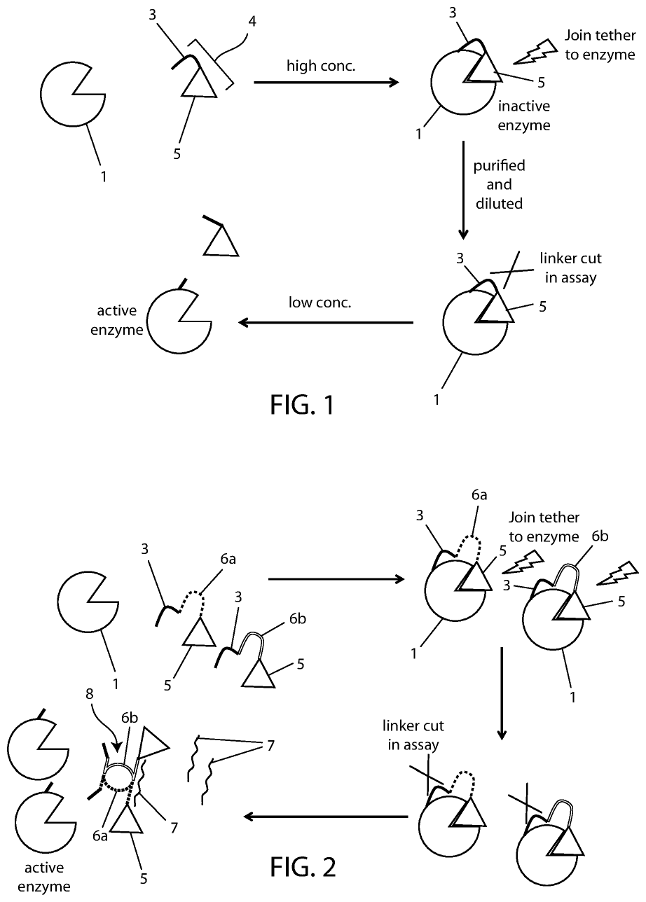

For example, FIG. 1 shows a schematic of an exemplary first embodiment of the invention, showing the assembly of the inhibited enzyme complex and the de-inhibition of the enzyme in an assay. To form the inhibited enzyme complex, the enzyme (1) is mixed with the inhibitor complex (4) comprising an inhibitor portion (5) and a linker portion (3), whereupon as least the inhibitor portion (5) of the inhibitor complex (4) will self-assemble to the enzyme (1). The concentration of the enzyme and inhibitor complex in this mixture is high enough to ensure that a high proportion of the enzyme will be associated with an inhibitor complex. Once the association is at the desired point an optional external stimulus can be applied to cause the tethering groups to join to the enzyme, thus anchoring the inhibitor complex in place. After formation of the inhibited enzyme complex, it can be purified of extraneous species, such as unbound inhibitor complex, uninhibited enzyme or other reactants added as part of the formation process, etc. When the linkers are cleaved in the assay, the cleaved inhibitor complex will dissociate to a substantial extent from the enzyme due to the dilution of the inhibited enzyme complex, de-inhibiting the enzyme in the process. In this context substantial dissociation means that the cleaved inhibitor complex is dissociated sufficiently that the enzyme can produce an acceptably large amount of signal generating species in the presence of the analyte of interest, over the range of analyte concentration of interest.

According to a second embodiment of the invention disclosed herein, an amplifying portion can be incorporated into the linker portion of the inhibitor complex. For example, the amplifying portion can be a sequence of DNA that can function as a DNAzyme, or as a partzyme that can join with a second partzyme and a facilitator to form an MNAzyme, as is known in the prior art. The DNAzyme or MNAzyme is capable of cleaving the linking portion of the enzyme complex under the correct conditions. For example, DNAzymes require the presence of divalent cations such as calcium or magnesium to be able to cleave their substrate and partzymes require the presence of the complimentary partzyme and facilitator DNA as well as calcium or magnesium to function. By ensuring that the conditions necessary for the DNAzyme or partzyme portion of the linker to be able to cleave substrate are not present during the preparation of the inhibited enzyme complex, premature cleavage of the linker portions of the inhibitor complex can be avoided.

The amplifying portion of the linker can be positioned such that the linker cleavage point is between the end of the amplifying portion distal to the inhibitor portion and the point at which the linker is joined to the enzyme. In this way, when the linker is cleaved, the amplifying portion will be liberated along with the inhibitor portion. If the amplifying portion comprises a DNAzyme, once liberated, it is free to fold into the conformation that can cleave the linker portion of the inhibitor complex. If the amplifying portion comprises a partzyme, once liberated, it can combine with the complimentary partzyme and facilitator DNA to form an MNAzyme that can cleave the linker portion of the inhibitor complex. The complimentary partzyme and/or the facilitator DNA can be present as free species in the assay solution, or one or both of them can be incorporated as alternative amplifying portions in additional enzyme inhibitor complexes in the assay mixture. This can be advantageous to suppress cleavage of the linker in the absence of the analyte of interest.

According to this embodiment, cleavage of the linker simultaneously de-inhibits the enzyme and generates further species capable of cleaving the linker. This leads to exponential growth in the rate of cleavage events occurring and thus the concentration of de-inhibited enzyme over time and so is capable of a cascading amplification of the original cleavage caused by the presence of the analyte.

For example, FIG. 2 shows a schematic of an exemplary second embodiment of the invention using Partzymes, showing the assembly of the inhibited enzyme complex with amplifying portions included in the linker (3), the de-inhibition of the enzyme (1) in an assay and formation of an amplification MNAzyme (8). An amplifying portion can be incorporated into the linker portion (3) of the inhibitor complex. For example, when the amplifying portion comprises a partzyme (6a), once liberated (e.g., by the linker being cut in an assay by initiator Partzymes and Analyte DNA), it can combine with the complimentary partzyme (6b) and facilitator DNA (7) to form an amplification MNAzyme (8) that can cleave more tethers to create a cascade.

According to a third embodiment of the invention disclosed herein, the inhibitor complex according to the second embodiment can be simplified and is not bound to or does not inhibit an enzyme. According to this embodiment, the inhibitor complex can be replaced with an amplifier/linker complex which is bound to a carrier that serves to prevent the amplifying portion from being active in the bound state. The carrier can be anything that prevents the amplifying portion of the inhibitor complex from being active. Examples of suitable carriers, include, but are not limited to, macromolecules such as proteins or other polymers that are soluble in the assay fluid; beads, rods or other shapes comprising polymers that are insoluble in the assay fluid such as latex or polystyrene beads, magnetic beads, or other species that are typically larger than the inhibitor complex. It is preferred that the carrier be small enough that it can be freely dispersed and is highly mobile in the assay solution and has a large surface area to allow facile interaction with any cleaving constructs present. The carrier can also be capable of forming either a covalent or non-covalent bond with the tethering groups of the amplifier/linker complex. According to this embodiment, it is preferable that the amplifier/linker complex is tethered at at least two points to help ensure that the amplifying portion of the amplifier/linker complex is not active. The tethering can be accomplished using the methods disclosed as part of the first and second embodiments. Additionally, in this embodiment, pre-existing binding pairs can be utilised to achieve tethering. For example, the carrier can for example be streptavidin and the tethering group biotin, whereby upon mixing the biotin on the amplifier/linker complex binds to the biotin binding sites of the streptavidin, thus tethering the inhibitor complex to the carrier.

According to this embodiment, it is not necessary to have a portion in the complex that associates strongly with the carrier. For example, two linker portions comprising amplifying portions can be joined directly together or via a small linking molecule to form an amplifier/linker complex. Optionally, the inhibitor portion can be replaced with a portion that aids association of the amplifier/linker complex to the carrier by being attracted to the carrier, here termed a passive associator, but it is not required to inhibit the action of the carrier in any way. Collectively, the inhibitor portion or passive associator are here termed the assembly facilitator portion of the inhibitor or amplifier/linker complex. The added flexibility associated with this embodiment can assist in finding species that have all the required characteristics to practice the invention disclosed herein.

According to this embodiment, the exponential growth in the concentration of cleavage agents proceeds as it does in the second embodiment, but in this embodiment, the cleavage agents generate a readable signal by cleaving a separate signal generation species. The separate signal generation species can be the inhibited enzyme complex according to the first embodiment. Alternatively, it can be another species, such as a substrate for a DNAzyme or MNAzyme that comprises a fluorescent group and a quencher group such as is known in the prior art, where the fluorescent group begins to fluoresce when the substrate is cleaved to separate it from the quencher group. This embodiment can be useful, for example, if it is difficult to satisfy all the required characteristics of the second embodiment disclosed herein simultaneously. This embodiment allows the required characteristics for the inhibitor complex comprising an amplifying portion to be optimised separately from the characteristics required to generate a readable signal.

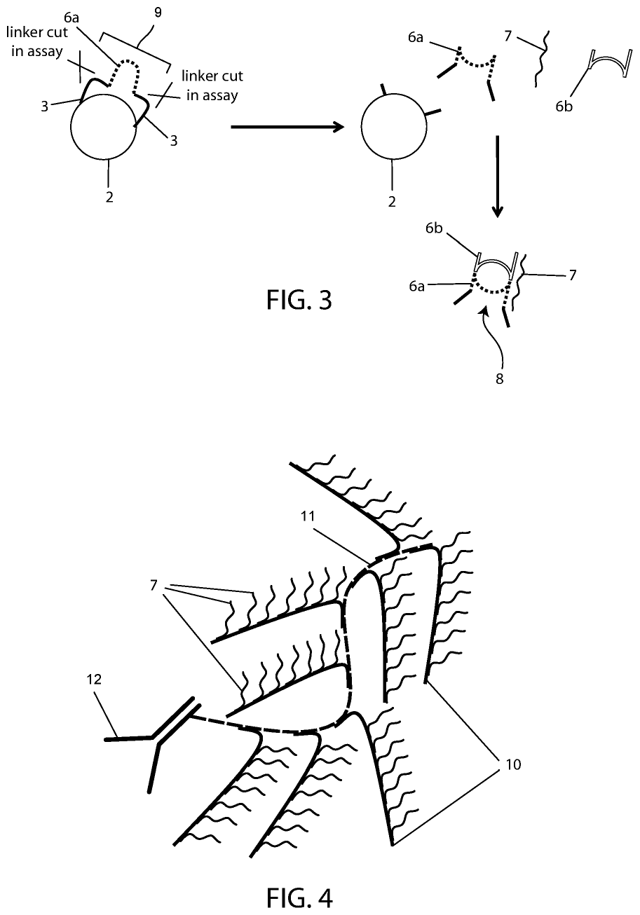

For example, FIG. 3 shows a schematic an exemplary third embodiment of the invention using Partzymes, wherein a Partzyme (6a) is part of the linker joined to a carrier (1), showing the formation of an amplification MNAzyme (8). An amplifier/linker complex (9) is bound to a carrier (2) that serves to prevent the amplifying portion from being active in the bound state. The amplifier/linker complex (9) including a Partzyme (6a) can be tethered at at least two points to help ensure that the amplifying portion of the amplifier/linker complex is not active. When cleaved, the amplifier/linker complex (9) disassociates and can, for example, join with a facilitator DNA (7) and second Partzyme (6b) in reagent. The exponential growth in the concentration of cleavage agents proceeds as it does in the second embodiment, but in this embodiment the cleavage agents generate a readable signal by cleaving a separate signal generation species. The separate signal generation species can be the inhibited enzyme complex according to the first embodiment of this invention.

In a fourth embodiment, a method is disclosed whereby the first, second and third embodiments of the invention can be used to detect the presence of species that can specifically participate in a binding event. This embodiment is suitable for any analyte that can be selectively bound to a second moiety or binding partner. Well known selective binding partners in the prior art include antigens and antibodies, where the antigens are typically proteins, however, any case where one binding partner is the analyte of interest and binds selectively to a binding partner is suitable for use herein. For example, the binding partners can be complementary strands of DNA, where one of the strands is the analyte of interest. In some embodiments, DNA can be immobilised on a surface, for example the surface of a magnetic bead or gold electrode, wherein the immobilised DNA can bind to a first portion of an analyte strand of DNA and the binding moiety that comprises a labeled binding complex can bind to a second portion of an analyte of DNA, whereby the analyte strand of DNA forms a bridge between the immobilised DNA and the labeled binding complex. For the purposes of illustration, in disclosing this embodiment of the invention, one of the binding partners will be referred to as the analyte and the other (the labeled binding complex) as a labelled antibody (L-Ab). This embodiment is applicable to competitive assays, where the analyte competes with an immobilised binding partner to bind to the antibody. It is also applicable to sandwich assays, where the analyte causes a sandwich to form between a capture antibody and labelled antibody. In the competitive assay, an increased amount of analyte causes an increased amount of L-Ab to be free in the solution. In this type of assay, the free L-Ab is separated from the immobilised L-Ab and the label on the free L-Ab used to generate a signal. In a sandwich assay, an increased amount of analyte causes an increased amount of L-Ab sandwiches to form. In this assay, the sandwiches can be formed on a structure such as magnetic beads, where the bound L-Ab is separated from the free L-Ab and the label on the bound L-Ab used to generate a signal. This embodiment is particularly suitable when the labeled binding moiety is a DNA strand that is joined to an immobilised DNA strand via an analyte DNA strand.

According to this embodiment, the label on the antibody comprises species that can directly cause cleavage of a substrate or are part of a construct that can cause cleavage of a substrate. Preferably, the label comprises at least one DNA sequence, here termed cleaving DNA, that can directly or indirectly cause a substrate to be cleaved. A further preferred embodiment is where the label comprises multiple copies of the cleaving DNA. A further preferred embodiment is where the label comprises multiple copies of cleaving DNA that can function as a DNAzyme. A most preferred embodiment is where the label comprises multiple copies of cleaving DNA that can function as facilitator DNA that can hybridise to partzymes to form functional MNAzymes.

A label comprising one or many copies of cleaving DNA can be formed by joining the one or more cleaving DNA copies directly to the antibody, or preferably by hybridising one or more cleaving DNA copies to a backbone DNA that is joined to the antibody or more preferably, hybridising multiple copies of the cleaving DNA to DNA branches that are covalently linked to a backbone that is joined to the antibody, or most preferably that multiple copies of the cleaving DNA are hybridised to DNA branches that are in turn hybridised to backbone DNA that is joined to the antibody. Optionally, when a backbone is used, more than one antibody can be joined to the backbone. This can increase the avidity of binding of the L-Ab to the immobilised capture antibody in the presence of a given concentration of analyte. An advantage of the most preferred embodiment is that a large number of cleaving DNA copies can be incorporated into the L-Ab without having to construct complicated branched sequences of DNA. According to the most preferred embodiment, the DNA branches self-assemble to the DNA backbone and the cleaving DNA copies self-assemble to the DNA branches via DNA hybridisation, simply by being mixed together.

If the cleaving DNA is hybridised to a DNA branch or backbone, the complementary DNA sequences are preferably designed such that the cleaving DNA can be separated from the branch or backbone DNA as part of the signal generation process. This can be done for example by raising the temperature as part of the signal generation process sufficient to melt the cleaving DNA from the branch or backbone DNA. More preferably a complimentary DNA sequence can be provided as part of the signal generation process that binds to a section of the cleaving DNA that comprises the section of the cleaving DNA that binds to the branch or backbone DNA, whereby the complementary DNA binds sufficiently strongly to the cleaving DNA to strip it from the branch or backbone DNA and where the binding of the complementary DNA does not inhibit the cleaving function of the cleaving DNA. Most preferably, the cleaving DNA is facilitator DNA for an MNAzyme and the complementary DNA is one or both partzymes that bind to the facilitator DNA to strip it from the branch or backbone DNA. MNAzymes can then be formed simultaneously with the stripping process. To facilitate the stripping process it is preferable that the cleaving DNA is hybridised to the branch or backbone DNA such that at least one end of the cleaving DNA is not bound and can function as an initial attachment site for the stripping complementary DNA.

For example, FIG. 4 shows an exemplary fourth embodiment of the invention, showing multiple facilitator DNA copies (7) hybridized to branch (10) and backbone DNA (11), with the backbone DNA joined to an antibody (12).

### Cleavage Initiation

In the embodiments disclosed herein, cleavage is initiated via the presence of the analyte of interest. According to the first embodiment of the invention, an initial cleaving agent generated via the analyte is the only one that is necessary to be present.

According to the second and third embodiments of the invention, the initial cleaving agent initiates a cascading generation of additional cleaving agents. The nature of the initial cleaving agent depends upon the analyte of interest. If the analyte of interest has its own cleavage action, for example a protease enzyme such as thrombin or trypsin, then the analyte itself can act as the initial cleaving agent. If the analyte is for example a specific sequence of DNA or RNA, then the initial cleaving agent can be an MNAzyme, where the facilitator for the MNAzyme is the analyte DNA or RNA sequence of interest and the thus formed MNAzyme can cleave the linker portion of the inhibitor complex or amplifier/linker complex.

According to the fourth embodiment of the invention, the analyte of interest causes facilitator DNA and/or DNAzymes to be present in the assay mixture. The facilitator DNA can combine with partzymes to from MNAzymes that can generate a signal via cleavage, as described in the other embodiments of the invention. As well as or alternatively, DNAzymes can generate a signal via cleavage as described in the other embodiments of the invention. According to the fourth embodiment, the analyte of interest can be a protein, DNA or other molecule that causes a binding event to happen.

Devices Suitable for Use with the Invention Methods

There are multiple devices that are suitable for use with some or all the disclosed molecular entities and methods. To practice the first, second or third embodiments of the invention, at least one reaction chamber can be used. The assay reagent can comprise the inhibited enzyme complex, the amplifier/linker complex, optional additional components to help form the cleaving agent, for example partzymes, a substrate for the enzyme and optionally mediators for the enzyme or other species such as those disclosed above to create a device readable signal. If the analyte has its own cleavage action, then the additional cleaving agent components may not be required. If the enzyme is capable of directly forming a readable signal species, then additional mediators may not be required. The reagents can initially be present in solution form or in dry form or with some in solution form and some in dry form. A solution containing the analyte can then be added to the reaction chamber to initiate the assay.

The reaction chamber can be in the form of a conventional tube or cuvette, a microfluidic space or other suitable form. The form can be tailored to the readout mechanism. For example, if an optical readout method is used, a cuvette may be the most suitable form. If an electrochemical readout method is employed, a microfluidic chamber comprising at least two electrodes can be used.

To practice the fourth embodiment of the invention, one or two reaction chambers can be used. If using one chamber, the analyte containing solution can be first mixed with reagents to cause the binding partners to bind. The bound, labelled binding partner is then separated from the free, labelled binding partner and the labelled binding partner remaining in the chamber assayed by addition of the detection reagents. The detection reagents comprise the reagents necessary to use the cleavage detection methods disclosed herein for this embodiment.

If two reaction chambers are used, then the binding reaction can proceed in one chamber and the detection reactions in a second chamber. This can be advantageous as it can facilitate separation of the bound and free labelled binding partner. For example, in a first chamber the immobilised binding partner and the labelled binding partner can be dried or be present in liquid form. A solution containing the analyte can be added to this chamber and any binding reactions allowed to proceed. After any binding had occurred, if desired to detect the remaining free labelled binding partner, an aliquot of the supernatant liquid can be transferred to a second chamber for detection. If desired to detect the bound labelled binding partner, then the material comprising the immobilised binding partner can be collected and transferred to a second chamber. If the remaining free labelled binding partner is to be detected, the immobilised binding partner can be immobilised on a surface of the first chamber or on a surface that can be separated from the supernatant in the first chamber, for example on polymer or magnetic particles. If the immobilised labelled binding partner is to be detected, the immobilised binding partner can be immobilised on a surface that is transferable to the second chamber, for example the surface of polymer or magnetic particles. If using polymer particles, they can be collected and washed, to remove supernatant containing free labelled binding partner, by using filtration or centrifugation for example. If using magnetic particles, they can be collected and washed, to remove supernatant containing free labelled binding partner, by using a magnetic field for example. The collected and washed particles can be transferred to the second chamber by being aspirated into a transfer device such as a pipette, or moved between chambers using other means, such as liquid flow that is pumped or created by capillary flow, or by the suitable application of magnetic fields in the case of magnetic particles.

The second chamber can contain the detection reagents in dry or liquid form. The detection reagents comprise the reagents necessary to use the cleavage detection methods disclosed here for this embodiment of the invention.

The embodiments of the invention described above can be combined in many different ways to confer utility in many applications. In order to set out more clearly examples of possible applications, components utilised in different embodiments of the invention are given a code and a table provided that lists the coded elements that can be combined to satisfy the requirements of the stated application area. These are presented as examples of the utility combinations of embodiments of the invention and are not intended to limit the scope of the invention.

For clarity, the table below is also provided to explain the meaning and function of the component types listed.

TABLE 1

| Component Type | Meaning and function |
|---|---|
| Linking portions of the | This is the portion of the inhibitor or plain carrier |
| inhibitor complex | associated complex that forms the cleavable link |
| | between the complex and the carrier. It is intended |
| | that the linker be cleaved during to assay if the |
| | analyte is present |
| Assembly Facilitator portion | This is the portion of the inhibitor or amplifier/linker |
| of the inhibitor complex | complex that helps to associate the complex with the |
| | carrier prior to tethering the complex to the carrier. |
| | In the case of an inhibitor complex it also functions |
| | to inhibit the action of the enzyme in reacting with |
| | its substrate. For an amplifier/linker complex it may |
| | be optional if the remaining portions of the complex |
| | associate with the carrier sufficiently well to allow |
| | the tethering to occur. |
| Carrier for the inhibitor | This is the species to which the inhibitor or |
| complex | amplifier/linker complex associates. It is either an |
| | enzyme of other carrier. If amplifying portions are |
| | present in the inhibitor or amplifier/linker complex it |
| | also functions to inhibit the action of the amplifying |
| | portions. |
| Signal Generator | This is the species plus device to be used to generate |
| | a device readable signal from the species produced in |
| | response to the presence of the analyte. |
| Source of assay Initiator | This is the species that generates the primary |
| | cleavage events, that is, the events that initiate |
| | cleavage reactions occurring in the assay |
| Assay Format | This is the basic assay format that can be suitable for |
| | use with the listed combination of components. |

### Components

### Linking Portions of the Inhibitor Complex

T1—MNAzyme or DNAzyme substrate
T2—MNAzyme or DNAzyme substrate+amplifying portion

### T3—Mixture of T1 and T2

T4—Substrate for other enzyme with cleavage action
T5—Substrate for other enzyme with cleavage action+MNAzyme or DNAzyme substrate+amplifying portion

### Assembly Facilitator Portion of the Inhibitor Complex

A1—Enzyme inhibitor
A2—Plain carrier associator or no associator or inhibitor

### Carrier for the Inhibitor Complex

C1—Enzyme that can generate as least one electroactive species
C2—Plain carrier macromolecule
C3—Other signal generating enzyme

### Signal Generator

S1—Electroactive species+electrodes and electrical detection device
S2—Coloured species+optical detector
S3—Cleavage of fluorophore/quencher+fluorescence detector

### Source of Assay Initiator

I1—Analyte DNA activating initiator partzymes
I2—Facilitator DNA hybridized to an Ab/DNA conjugate
I3—Analyte enzyme with cleavage action in sample

### Assay Format

F1—All liquid well or cuvette type format with multiple liquid change steps. All the reagents used can be liquids in this format
F2—External liquid pre-treatment/binding steps with dry detection reagents. In this format some pre-treatment of the sample is done in the liquid phase external to the detection chamber and initially dry detection reagents are used in the detection chamber
F3—Internal to device liquid pre-treatment with dry detection. In this format any sample pre-treatment is done in the liquid phase and is integrated into the same device that comprises the detection chamber with initially dry detection reagents
F4—Fully dry single chamber detection. The device can only require a single chamber with initially dry detection reagents

### Applications

The codes above are used below in combination to specify examples of what combinations can be useful for the application listed.

TABLE 2

| | Type of | | Assay configuration/ | |
|---|---|---|---|---|
| Application | test | Readout | Advantages | Combination |
| DNA test | Simple | Electrochemical | Very fast non PCR | T2/A1/C1/S1/I1/F3 |
| | POC | | DNA/RNA sequence | |
| | | | detection with single step | |
| | | | disposable | |
| DNA test | Moderately | Electrochemical | Liquid pre- | T2/A1/C1/S1/I1/F2 |
| | complex | | treatment/extraction kit | |
| | POC or | | with dry disposable | |
| | Lab | | detection | |
| DNA test | Lab test | Colourometric | Very fast non PCR | T2/A1/C1/S2/I1/F1 |
| | | | DNA/RNA sequence | |
| | | | detection using liquid | |
| | | | reagents | |
| DNA test | Lab test | Fluorescent | Very fast non PCR | T2/A2/C2/S3/I1/F1 |
| | | | DNA/RNA sequence | |
| | | | detection using liquid | |
| | | | reagents | |
| DNA test | Moderately | Fluorescent | Fast non PCR dry | T2/A2/C2/S3/I1/F2 |
| | complex | | detection reagent | |
| | POC or | | DNA/RNA sequence | |
| | Lab | | detection with | |
| | | | fluorescence readout | |
| | | | with reagents dried into a | |
| | | | cuvette | |
| Moderate to | Simple | Electrochemical | Microfluidic POC | T1/A1/C1/S1/I2/F3 |
| high | POC | | immunoassay | |
| sensitivity | | | | |
| immunoassay | | | | |
| Ultra-high | Simple | Electrochemical | Microfluidic POC | T3/A1/C1/S1/I2/F3 |
| sensitivity | POC | | immunoassay with | |
| immunoassay | | | partial to full cascade | |
| | | | amplification | |
| Ultra-high | Moderately | Electrochemical | Immunoassay with | T3/A1/C1/S1/I2/F2 |
| sensitivity | complex | | liquid pre-treatment with | |
| immunoassay | POC or | | partial to full cascade | |
| | Lab | | amplification and dry | |
| | | | detection strip | |
| Ultra-high | Moderately | Colourometric | Immunoassay with | T3/A1/C1/S2/I2/F2 |
| sensitivity | complex | | liquid pre-treatment with | |
| immunoassay | POC or | | partial to full cascade | |
| | Lab | | amplification and dry | |
| | | | detection cuvette | |
| Ultra-high | Lab test | Colourometric | ELISA type assay with | T3/A1/C1/S2/I2/F1 |
| sensitivity | | | ultra-sensitive detection | |
| immunoassay | | | for low | |
| | | | concentration/fast test | |
| | | | time assay | |
| Ultra-high | Lab test | Fluorescent | ELISA type assay with | T3/A2/C2/S3/I2/F1 |
| sensitivity | | | ultra-sensitive detection | |
| immunoassay | | | for low | |
| | | | concentration/fast test | |
| | | | time assay | |
| Ultra-high | Moderately | Fluorescent | Immunoassay with | T3/A2/C2/S3/I2/F2 |
| sensitivity | complex | | liquid pre-treatment with | |
| immunoassay | POC or | | partial to full cascade | |
| | Lab | | amplification and dry | |
| | | | detection cuvette | |
| Ultra-high | Simple | Fluorescent | Microfluidic with liquid | T3/A2/C2/S3/I2/F3 |
| sensitivity | POC | | wash with partial to full | |
| immunoassay | | | cascade amplification | |
| | | | and dry detection cuvette | |
| Moderate to | Simple | Electrochemical | Microfluidic assay | T4/A1/C1/S1/I3/F3 |
| high | POC | | format for enzymes with | |
| sensitivity | | | a cleavage action | |
| cleavage | | | | |
| enzyme | | | | |
| assay | | | | |
| Ultra- high | Simple | Electrochemical | Microfluidic assay | T5/A1/C1/S1/I3/F3 |
| sensitivity | POC | | format for enzymes with | |
| POC | | | a cleavage action using | |
| cleavage | | | cascade amplification | |
| enzyme | | | | |
| assay | | | | |
| Moderate to | Moderately | Electrochemical | Liquid pre-treatment step | T4/A1/C1/S1/I3/F2 |
| high | complex | | with dry strip detection | |
| sensitivity | POC or | | | |
| cleavage | Lab | | | |
| enzyme | | | | |
| assay | | | | |
| Ultra- high | Moderately | Electrochemical | Liquid pre-treatment step | T5/A1/C1/S1/I3/F2 |
| sensitivity | complex | | with dry strip detection | |
| cleavage | POC or | | | |
| enzyme | Lab | | | |
| assay | | | | |
| Moderate to | Moderately | Colourometric | Liquid pre-treatment step | T4/A1/C1/S2/I3/F2 |
| high | complex | | with dry cuvette | |
| sensitivity | POC or | | detection | |
| cleavage | Lab | | | |
| enzyme | | | | |
| assay | | | | |
| Ultra- high | Moderately | Colourometric | Liquid pre-treatment step | T5/A1/C1/S2/I3/F2 |
| sensitivity | complex | | with dry cuvette | |
| cleavage | POC or | | detection | |
| enzyme | Lab | | | |
| assay | | | | |
| Moderate to | Lab | Colourometric | Full liquid assay | T4/A1/C1/S2/I3/F1 |
| high | | | | |
| sensitivity | | | | |
| cleavage | | | | |
| enzyme | | | | |
| assay | | | | |
| Ultra- high | Lab | Colourometric | Full liquid assay | T5/A1/C1/S2/I3/F1 |
| sensitivity | | | | |
| cleavage | | | | |
| enzyme | | | | |
| assay | | | | |
| Moderate to | Lab | Fluorescent | Full liquid assay | T4/A2/C2/S3/I3/F1 |
| high | | | | |
| sensitivity | | | | |
| cleavage | | | | |
| enzyme | | | | |
| assay | | | | |
| Ultra- high | Lab | Fluorescent | Full liquid assay | T5/A2/C2/S3/I3/F1 |
| sensitivity | | | | |
| cleavage | | | | |
| enzyme | | | | |
| assay | | | | |

### EXAMPLES

Embodiments of the invention are exemplified and additional embodiments are disclosed in further detail in the following Example, which is not in any way intended to limit the scope of any embodiment of the invention described herein.

### Example 1

Construction of an inhibited enzyme complex.

TABLE 3
Description of molecular species.

| | |
|---|---|
| Inhibitor: reversible trypsin inhibitor, structure based on benzamidine with lysine to form the side chain | 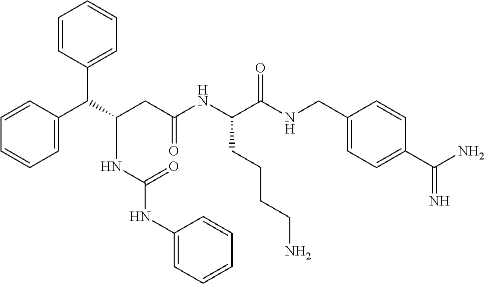 [SMILES: N=C(N)c1ccc(CNC(=O)[C@H](CCCCN)NC(=O)C[C@H](NC(=O)Nc2ccccc2)C(c2ccccc2)c2ccccc2)cc1] |
| Modified oligonucleotide (IDT ®): single stranded DNA/RNA bases oligo with a primary amine group on one end of the oligo and a disulphide group on the other end of | 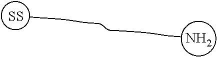 |
| the oligo | |
| PART A. Synthesis of the oligo-CL- inhibitor | 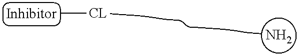 |
| PART B. Synthesis of the Trypsin-CL- oligo-CL-inhibitor | 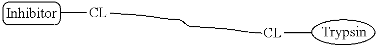 |

CL refers to crosslinker
Oligo refers to oligonucleotide

### PART A. Synthesis of the Oligo-CL-Inhibitor

Step 1: The primary amine group on the inhibitor was reacted with sulfo-SMCC (4-(N-Maleimidomethyl)cyclohexane-1-carboxylic acid 3-sulfo-N-hydroxysuccinimide ester sodium salt, SIGMA-ALDRICH®) to form a maleimide. The inhibitor was in excess to ensure all NHS groups on sulfo-SMCC are blocked after completion of the reaction.

Step 2: The disulphide bond on the modified oligo (IDT) was reduced to a thiol using an excess of TCEP (Tris(2-carboxyethyl)phosphine hydrochloride, SIGMA-ALDRICH®).

Step 3: The thiol terminated oligo was reacted with a small excess of maleimide activated inhibitor to ensure most of the oligos were crosslinked to an inhibitor molecule.

Step 4: The oligo-CL-inhibitor conjugate was purified using Nanosep® Centrifugal device 3K (Pall Corp.) to remove free unreacted species and side-reaction products. Low MW materials were removed in the filtrates whereas the conjugate remained in the sample reservoir as it did not pass through the membrane.

### PART B. Synthesis of the Trypsin-CL-oligo-CL-inhibitor

Step 1: Trypsin (Trypsin from porcine pancreas, SIGMA-ALDRICH®) was mixed with the purified oligo-CL-inhibitor conjugate at a concentration and ratio where the activity of the enzyme is fully inhibited.

Step 2: EDC (1-ethyl-3-(3-dimethylaminopropyl)carbodiimide hydrochloride, Thermo Fisher Scientific Inc.) was added to the mixture to activate available carboxyl groups on the trypsin for direct reaction with the terminal primary amine of the modified oligo via amide bond formation. This formed an inhibited enzyme complex.

Step 3: The inhibited enzyme complex was purified using a Nanosep® Centrifugal device 30K (Pall Corp.) to remove free unreacted species and side-reaction products. The resulting product was ready for use in an assay.

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law), regardless of any separately provided incorporation of particular documents made elsewhere herein.

The use of the terms “a” and “an” and “the” and similar referents in the context of describing the invention are to be construed to cover both the singular and the plural, unless otherwise indicated herein or clearly contradicted by context.

Unless otherwise stated, all exact values provided herein are representative of corresponding approximate values (e.g., all exact exemplary values provided with respect to a particular factor or measurement can be considered to also provide a corresponding approximate measurement, modified by “about,” where appropriate). All provided ranges of values are intended to include the end points of the ranges, as well as values between the end points.

The description herein of any aspect or embodiment of the invention using terms such as “comprising”, “having,” “including,” or “containing” with reference to an element or elements is intended to provide support for a similar aspect or embodiment of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

All headings and sub-headings are used herein for convenience only and should not be construed as limiting the invention in any way.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The citation and incorporation of patent documents herein is done for convenience only and does not reflect any view of the validity, patentability, and/or enforceability of such patent documents.

This invention includes all modifications and equivalents of the subject matter recited in the claims and/or aspects appended hereto as permitted by applicable law.

1. A molecular entity comprising:
a. an enzyme, and
b. an enzyme inhibitor complex;
wherein the enzyme inhibitor complex comprises an inhibitor portion linked to a linker portion;
wherein the linker portion is capable of being tethered to the enzyme and capable of being cleaved by a cleaving agent;
wherein when the linker portion is tethered to the enzyme, activity of the enzyme is inhibited;
wherein when the linker portion is cleaved, activity of the enzyme is increased.

2. The molecular entity of claim 1 wherein the linker portion comprises a cleavage site, a distal side and a medial side, wherein the medial side and distal side are at opposite sides of the cleavage site, wherein the inhibitor portion is linked to the linker portion at a position on the medial side and the linker portion is capable of being linked to the enzyme by a reactive species at a position on the distal side.

3. The molecular entity of claim 1 wherein the linker portion comprises at least two cleavage sites, a middle region and at least two sides distal to the middle region, wherein a cleavage site is positioned between each distal side and the middle region, wherein the inhibitor portion is linked to the linker portion in the middle region and the linker portion is capable of being linked to the enzyme by a reactive species at a position on the distal sides.

4. The molecular entity of claim 2 wherein the reactive species is capable of forming a covalent bond with the enzyme either directly or via a crosslinking agent.

5. The molecular entity of claim 2 wherein the distal end of the linker portion links to the enzyme via the application of UV light, a crosslinking agent, an activating agent, catalyst or heat.

6. The molecular entity of claim 2 wherein the inhibitor portion associates with the enzyme at a concentration of enzyme inhibitor complex mixed with a concentration of enzyme; wherein the concentrations are high compared to the concentration of inhibited enzyme complex to be used in an assay reagent.

7. The molecular entity of claim 1 wherein the enzyme is a redox enzyme or a cleavage enzyme.

8. The molecular entity of claim 1 wherein the enzyme inhibitor complex comprises a linker amplifying portion.

9. The molecular entity of claim 8 wherein the linker portion is capable of being cleaved by a cleaving agent, wherein cleavage of the linker portion results in (i) activation of the enzyme and (ii) formation and activation of the linker amplifying portion that is capable of cleaving a second linker portion or when joining with other components is capable of cleaving a second linker portion.

10. The molecular entity of claim 8 wherein the linker amplifying portion forms a DNAzyme or one or more portions of an MNAzyme when activated.

11. The molecular entity of claim 1, wherein the cleaving agent is a target analyte and wherein the activity of the enzyme generates a readout signal.

12. A method for detecting a cleavage event in a molecule comprising:
a. providing the entity of claim 1 in a state wherein the linker portion is tethered to the enzyme and activity of the enzyme is inhibited;
b. subjecting a test sample to (a); and
c. detecting a device readable signal;
wherein the detection of a device readable signal is indicative of a cleavage event.

13. The method of claim 12 wherein the cleavage event is indicative of the presence of an analyte in the test sample.

14. The method of claim 12 wherein the entity of (a) is contained in a solution and or a test strip.

15. The method of claim 12 wherein the detecting step comprises detecting changes in electrical voltage, electrical current, optical absorbance, colour, fluorescence or chemiluminescence.

16.-20. (canceled)

21. A molecular entity comprising at least one binding moiety linked to one or more DNA strands; wherein at least one of the DNA strands comprises a DNAzyme or a component of an MNAzyme, wherein the one or more DNA strands can be dissociated from the molecular entity in the presence of complementary DNA and/or a temperature elevation in a detection chamber.

22. The molecular entity of claim 21 wherein a molecule binding to the binding moiety results in the molecular entity being present in the detection chamber.

23. The molecular entity of claim 21 wherein the one or more branch DNA strands is linked to the binding moiety via a DNA backbone.

24. The molecular entity of claim 21 wherein the binding moiety is an antibody or DNA strand.

25. The molecular entity of claim 21 wherein one or more branch DNA strands are linked to the DNA backbone and wherein DNAzymes or a component of MNAzymes are linked to the one or more branch DNA strands.

26.-27. (canceled)
